(19) United States
(12) Patent Application Publication
(10) Pub. No.: US 2024/0173150 A1
(43) Pub. Date: May 30, 2024

(54) ACTUATORS FOR POWERED PROSTHESES

(71) Applicant: The Board of Trustees of The University of Alabama, Tuscaloosa, AL (US)

(72) Inventors: Xiangrong Shen, Tuscaloosa, AL (US); MD Rayhan Afsar, Tuscaloosa, AL (US); MD Rejwanul Haque, Tuscaloosa, AL (US)

(21) Appl. No.: 18/523,252

(22) Filed: Nov. 29, 2023

Related U.S. Application Data
(60) Provisional application No. 63/428,561, filed on Nov. 29, 2022.

(51) Int. Cl. A61F 2/70

(52) U.S. Cl. CPC A61F 2/70

(57) ABSTRACT

Example actuators for powered prostheses and powered prostheses are described herein. An example actuator for a powered prosthesis includes a motor including a motor output shaft and connected to a motor output sprocket; an intermediate shaft comprising a first intermediate sprocket and a second intermediate sprocket; an output sprocket; a first drive chain configured to engage the first intermediate sprocket and the motor output sprocket; and a second drive chain configured to engage the second intermediate sprocket and the output sprocket.

### CROSS-REFERENCE TO RELATED APPLICATIONS

This application claims the benefit of U.S. provisional patent application No. 63/428,561 filed on Nov. 29, 2022, and titled “ACTUATORS FOR POWERED PROTHESES,” the disclosure of which is expressly incorporated herein by reference in its entirety.

### GOVERNMENT SUPPORT

This invention was made with government support under grant number 1351520 awarded by the National Science Foundation and grant number 1734501 awarded by the National Science Foundation. The government has certain rights in the invention.

### BACKGROUND

Prostheses are commonly used as artificial replacements for body parts.

As an example, lower limb prostheses include prostheses designed to assist individuals who are missing part or all of a leg (e.g., amputees). Lower limb prostheses can be configured to replace the missing portion of leg. For example, in an individual whose leg is amputated below the knee, the prostheses can be configured to work with the existing knee joint. Alternatively, in a user whose leg is amputated above the knee, a prosthesis can be configured to replace both the missing knee and ankle joints.

Lower limb prostheses can be powered or unpowered. In an unpowered prosthesis, the wearer of the prosthesis must move the prosthesis themselves. This can cause fatigue and/or pain to the wearer, which can become aggravated over long periods of time. Powered prostheses can reduce the pain and fatigue on the wearer by moving part or all of the prosthesis using one or more actuators. For example, a powered lower leg prosthesis can include powered knee and/or ankle joints, where the knee/ankle joints move the lower leg and foot, respectively to simulate normal motion.

Therefore, what are needed are systems and methods that overcome challenges in the art of prostheses, some of which are described above.

### SUMMARY

The device, systems and methods described in this disclosure provides an actuator that can be used in powered prosthetics. The actuator can be compact, low cost, lightweight, and easier to fabricate than alternative actuators.

According to one aspect, the present disclosure relates to an actuator for a powered prosthesis. The example actuator includes a motor with an output shaft, connected to a motor output sprocket: an intermediate shaft including a first intermediate sprocket and a second intermediate sprocket: an output sprocket: a first drive chain configured to engage the first intermediate sprocket and the motor output sprocket: and a second drive chain configured to engage the second intermediate sprocket and the output sprocket.

In some implementations, the actuator includes an actuator housing, where the motor is fixedly attached to the actuator housing.

In some implementations, the actuator housing includes a proximal end and a distal end, the proximal end being proximal to the output sprocket, where the distal end includes an attachment surface.

In some implementations, the output sprocket defines a first axis of rotation and the motor output sprocket defines a second axis of rotation, and where the first axis of rotation is coaxial with the second axis of rotation.

In some implementations, the motor is an exterior-rotor brushless DC (ER-BLDC) motor.

In some implementations, the output sprocket produces less than or equal to 80 newton meters of torque.

In some implementations, the proximal end includes a rotational joint assembly.

In some implementations, the actuator includes a dual-stage transmission.

In some implementations, the actuator is housed in a prosthesis.

In some implementations, the prosthesis is a prosthetic ankle.

In some implementations, the powered prosthesis is a prosthetic knee.

In some implementations, the actuator is less than 200 mm long.

In some implementations, the actuator has a range of motion greater than 100 degrees.

According to another aspect, the present disclosure relates to a powered prosthesis. The example power prosthesis includes a first limb section and a second limb section; an actuator coupled to the first limb section and the second limb section, the actuator including: a motor fixedly mounted inside the actuator housing, the motor including an output shaft: a motor output sprocket: an intermediate shaft including a first intermediate sprocket and a second intermediate sprocket: an output sprocket: a first drive chain configured to engage the first intermediate sprocket and the motor output sprocket: and a second drive chain configured to engage the second intermediate sprocket and the output sprocket.

In some implementations, the powered prosthesis includes an actuator housing.

In some implementations, the actuator housing includes a proximal end and a distal end, the proximal end being proximal to the output sprocket, where the distal end includes an attachment surface.

In some implementations, the output sprocket defines a first axis of rotation and the motor output sprocket defines a second axis of rotation, and where the first axis of rotation is coaxial with the second axis of rotation.

In some implementations, the motor is an exterior-rotor brushless DC (ER-BLDC) motor.

In some implementations, the output sprocket produces less than or equal to 80 newton meters of torque.

In some implementations, the proximal end includes a rotational joint assembly.

In some implementations, the actuator is a dual-stage actuator.

In some implementations, the first limb section includes a lower leg, and the second limb section includes a foot.

In some implementations, the first limb section includes a lower leg and the second limb section includes an upper leg.

In some implementations, the first limb section and the second limb section have a range of motion greater than 100 degrees.

In some implementations, the actuator is less than 200 mm long.

Other systems, methods, features and/or advantages will become apparent to one with skill in the art upon examination of the following drawings and detailed description. It is intended that all such additional systems, methods, features and/or advantages be included within this description and be protected by the accompanying claims.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1A illustrates a side view of an example actuator, according to one implementation of the present disclosure.

FIG. 1B illustrates a perspective view of the example actuator shown in FIG. 1A, according to one implementation of the present disclosure.

FIG. 2A illustrates a perspective view of an actuator housing that encloses the actuator shown in FIGS. 1A-1B.

FIG. 2B illustrates an exploded view of the actuator housing shown in FIG. 2A.

FIG. 3A illustrates a perspective view of an example prosthetic joint including the actuator and actuator housing shown in FIGS. 1A-2B.

FIG. 3B illustrates a side-view of the example prosthetic joint shown in FIG. 3A.

FIG. 3C illustrates a cutaway view of the example prosthetic joint shown in FIGS. 3A-3B.

FIG. 4 illustrates an example prosthetic foot including the prosthetic joint shown in FIGS. 3A-3C.

FIG. 5A illustrates a side view of the prosthetic joint shown in FIGS. 3A-3C in the fully extended position.

FIG. 5B illustrates a side view of the prosthetic joint shown in FIGS. 3A-3C in the fully bent position.

FIG. 6A illustrates an experimental implementation of a prosthetic leg using two prosthetic joints of the present disclosure.

FIG. 6B illustrates an experimental implementation of a prosthetic leg using two prosthetic joints of the present disclosure.

FIGS. 7A-7D illustrate an experiment performed on an example implementation of the present disclosure.

FIG. 8 illustrates experimental results for the example implementation of the present disclosure.

FIG. 9 is an example computing device.

FIG. 10A illustrates an example transmission system layout for an example implementation of the present disclosure.

FIG. 10B illustrates a side view of the transmission system shown in FIG. 10A.

FIG. 11A illustrates a bumper and stopper according to implementations of the present disclosure.

FIG. 11B illustrates a bumper structure and bumper screw according to implementations of the present disclosure.

FIG. 12 illustrates a fabricated clamshell housing with structural ribs, positioning holes, and locating pins, according to implementations of the present disclosure.

### DETAILED DESCRIPTION

The device, systems and methods described in this disclosure provides an actuator that can be used in powered prosthetics, including, for example, powered prosthetic knees and ankles. The actuator can be compact, low cost, lightweight, and easier to fabricate than alternative actuators.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

The present methods and systems may be understood more readily by reference to the following detailed description of preferred embodiments and the Examples included therein and to the Figures and their previous and following description.

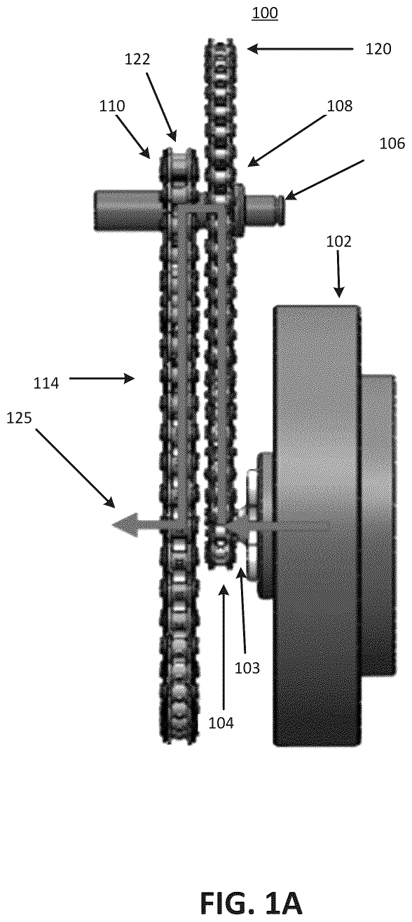

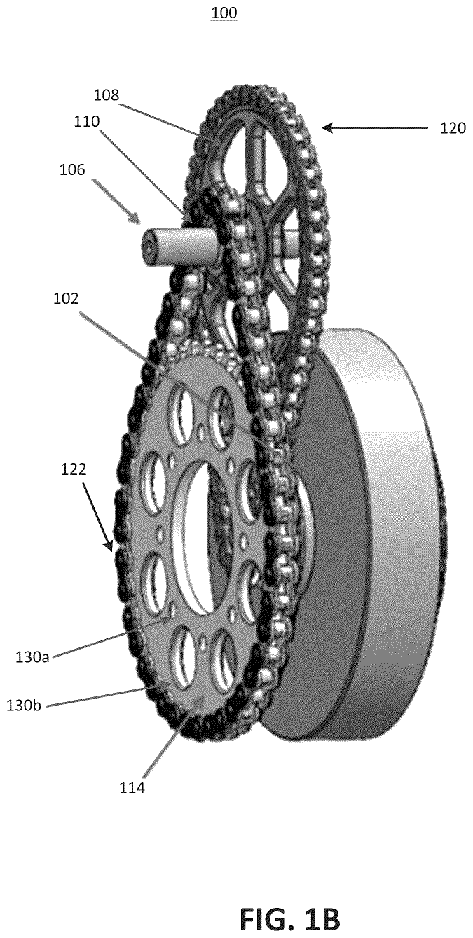

With reference to FIG. 1A, implementations of the present disclosure include actuators 100 for powered prostheses. The actuator 100 can include a motor 102 configured to drive a motor shaft 103, where the motor shaft 103 is connected to a motor output sprocket 104.

In some implementations, the motor is an exterior-rotor brushless DC (ER-BLDC) motor.

The actuator 100 can also include an intermediate shaft 106. The intermediate shaft 106 can include a first intermediate sprocket 108 and a second intermediate sprocket 110.

The actuator can output power through an output sprocket 114. The motor output sprocket 104 can be coupled to the first intermediate sprocket 108 by a first drive chain 120 so that the motor 102 can turn the intermediate shaft 106. The second intermediate sprocket 110 can be coupled to the output sprocket 114 by a second drive chain 122, so that power is transmitted from the motor 102 to the output sprocket 114. The power flow from motor to output sprocket 114 is shown in FIG. 1A with an arrow 125.

FIG. 1B illustrates a perspective view of the actuator 100 illustrated in FIG. 1A. As shown in FIG. 1B, the output sprocket 114 can include holes 130a for attaching the output sprocket 114 to an object (not shown) that the actuator 100 is configured to move. It should be understood that the number, size, and arrangement of the holes in FIG. 1B is provided only as an example. In some implementations, the output sprocket can include holes 130b to reduce the weight of the output sprocket. This disclosure contemplates providing an output sprocket having a different number, size, and/or arrangement of the holes.

As shown in FIG. 1B, the first drive chain 120 and second drive chain 122 can be arranged so that they are “folded” over one another. In other words, the output sprocket 114 and motor output sprocket 104 can have the same axis of rotation, or otherwise be close together, despite the motor output sprocket 104 and output sprocket 114 being connected through two drive chains 120 122.

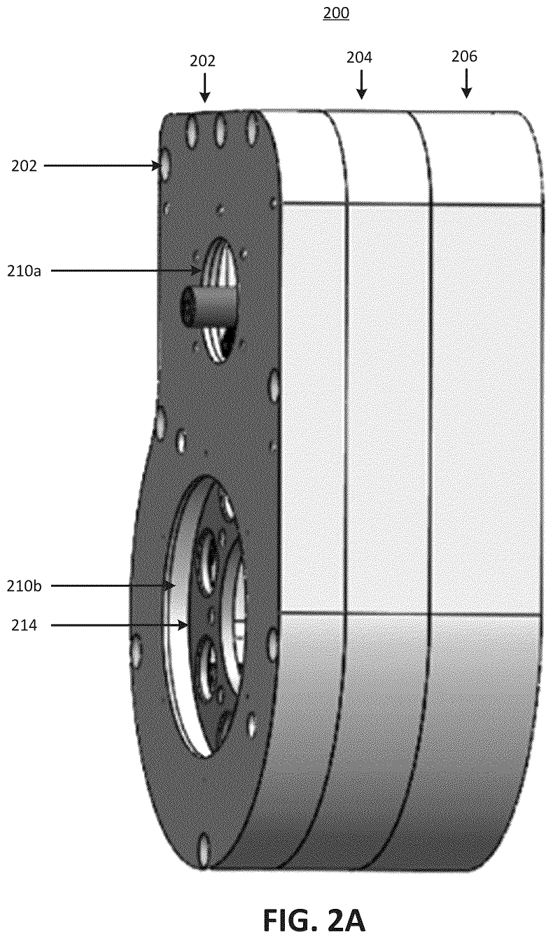

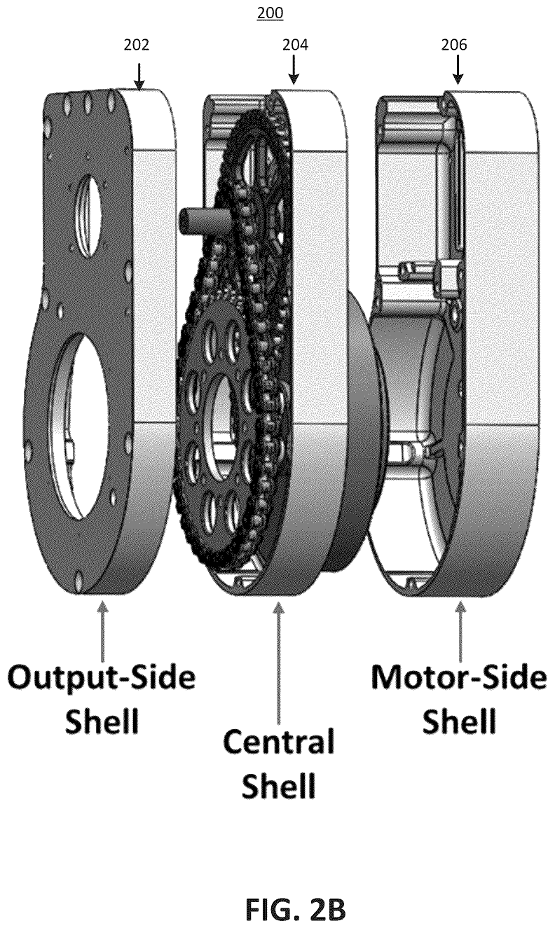

With reference to FIG. 2A-2B, the components shown in FIGS. 1A-1B can be enclosed by an actuator housing 200. The actuator housing 200 can be a load-bearing structure including one or more thin-walled shell-like components. Optionally, the actuator housing 200 can include three pieces, an output side shell 202, a central shell 204, and a motor side shell 206. Optionally, the motor side shell 206 can be configured to house the ER-BLDC motor, while the output side shell 202 and the central shell 204 form a hollow structure to accommodate the dual-stage folded-chain-drive actuator 100. It should be understood that the number, size, and arrangement of the shell pieces in FIGS. 2A-2B are provided only as an example. This disclosure contemplates providing an actuator housing having a different number, size, and/or arrangement of the shell pieces. The actuator housing 200 can optionally include cutouts 210a 210b to allow access to the output sprocket 214. As shown in FIG. 2A, the cutout 210b reveals the output sprocket and the cutout 210a reveals the intermediate shaft, which may be supported by a pair of bearings mounted in the housing. Additionally, the actuator housing 200 can include any number of holes or other attachment points on the surface of the actuator housing 200. Optionally, the holes can be used to attach the actuator housing 200 to a prosthesis.

FIG. 2B illustrates an exploded view of the actuator housing 200 shown in FIG. 2A, where the actuator 100 shown in FIG. 1A-1B can be seen inside the actuator housing 200.

The present disclosure contemplates different sizes of actuator housing 200. In some implementations, the actuator housing is less than 200 mm in its longest dimension. As a non-limiting example, an experimental implementation of the actuator housing is 160 mm in the longest dimension. The actuator housing 200 can be longer than 200 mm in some implementations, for example when the actuator is longer than 200 mm in the longest dimension. It should be understood that the dimensions of the actuator and actuator housing 200 described herein are intended only as non-limiting examples.

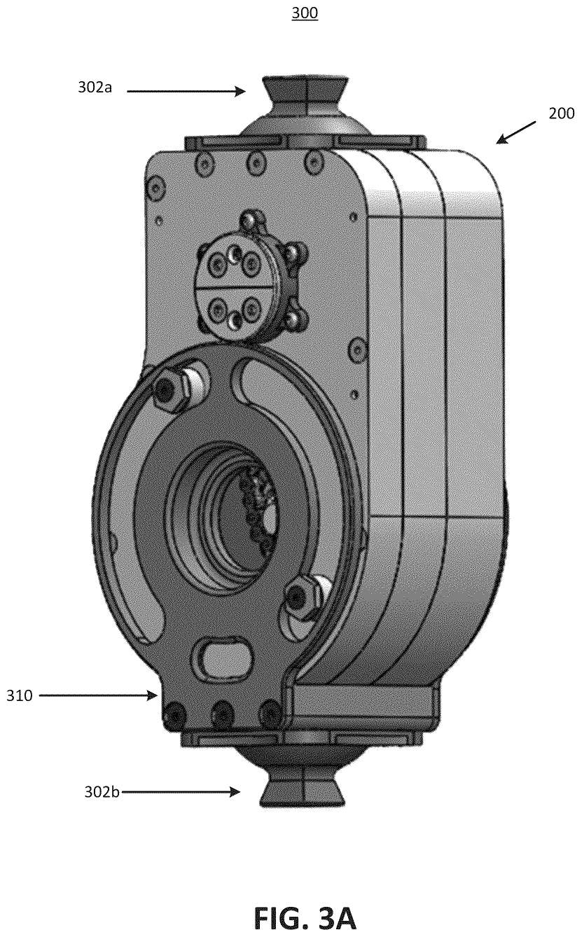

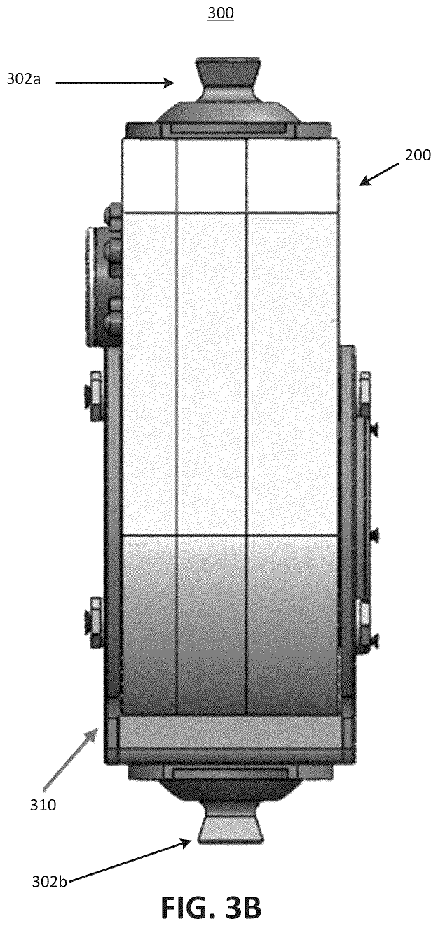

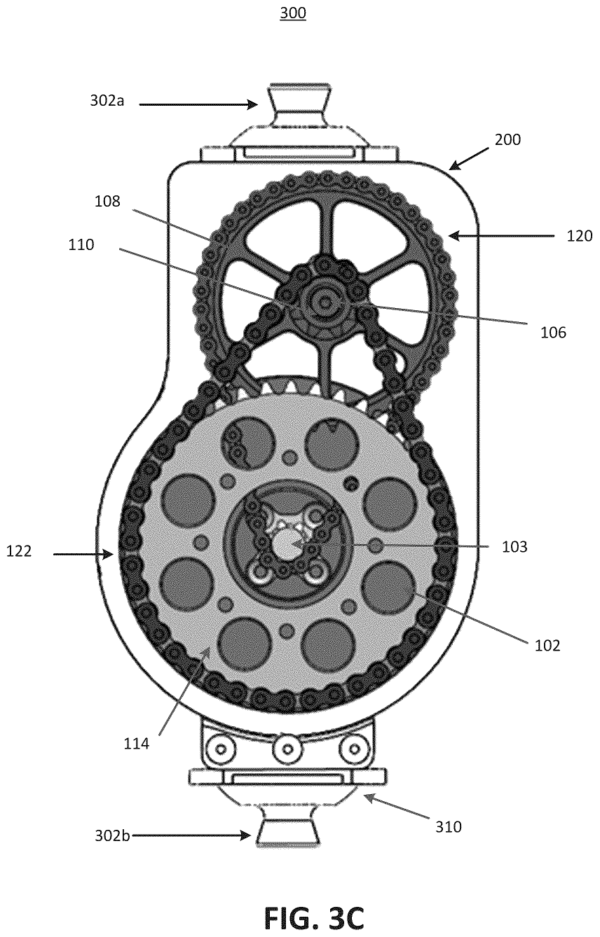

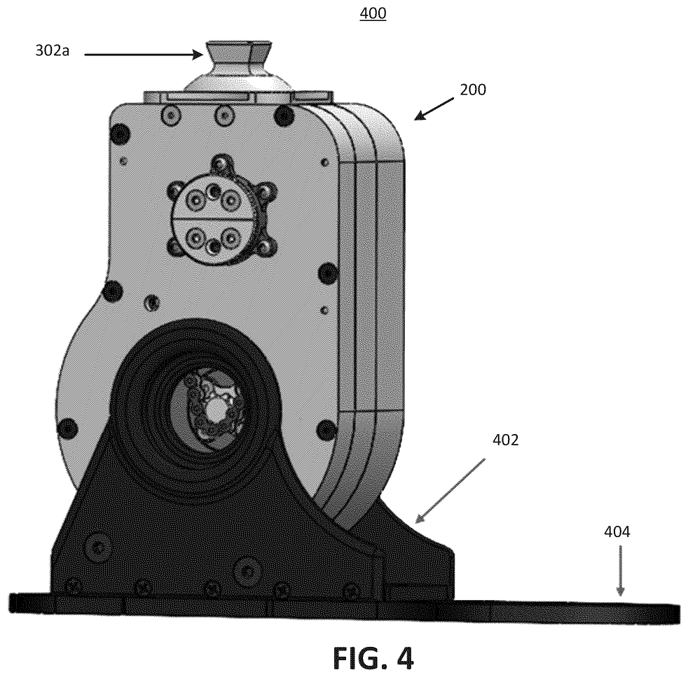

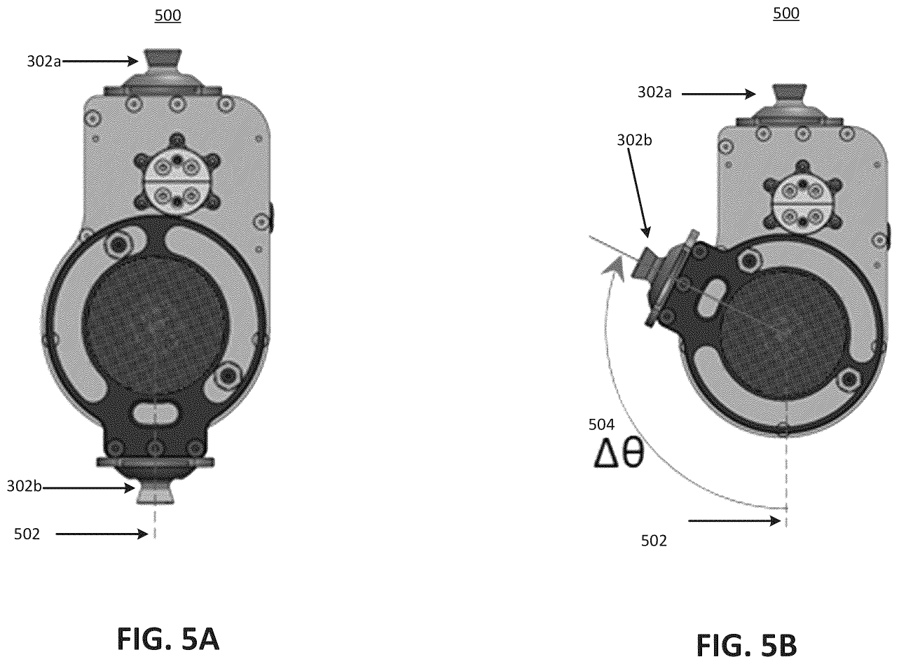

With reference to FIGS. 3A-3B, the actuator housing 200 shown in FIG. 2A-2B can be incorporated into a powered prosthetic joint 300. The powered prosthetic joint 300 can include connectors for attaching the powered prosthetic joint 300 to different portions of a prosthetic device, for example, the upper and lower parts of a prosthetic foot (see e.g., FIG. 4) or the upper and lower parts of a prosthetic knee (see e.g., FIGS. 5A-5B). Optionally, the connectors are pyramidal adapters 302a, 302b, which can be used to connect a variety of different configurations of prosthetics to the powered prosthetic joint 300. In some implementations, the powered prosthetic joint includes a single connector (see e.g., FIG. 4)), while in other implementations the powered prosthetic joint includes multiple connectors (see e.g., FIGS. 5A-5B).

The output sprocket (not shown) described in FIG. 1A and 1B, which is inside the actuator housing 200 can be connected to a rotational joint assembly 310.

For example, the output sprocket of the actuator can be connected to a fork-shaped Rotational Joint Assembly, which can form the rotational part of the prosthetic joint. The flat surface of the Rotational Joint Assembly also can serve as the mounting surface for the pyramid connector (configured as a knee prosthesis, FIGS. 5A-5B) or a prosthetic foot (configured as an ankle prosthesis, FIG. 4).

Thus, the torque output by the output sprocket can turn the rotational joint assembly 310 relative to the actuator housing 200. In turn, the pyramidal adapter 302b attached to the rotational joint assembly 310 turns. Thus, by moving the output sprocket, the angle between the pyramidal adapters 302a 302b can be changed.

Implementations of the present disclosure can be configured to produce less than or equal to 80 newton meters of torque on the rotational joint assembly 310. Alternatively or additionally, different transmission ratios and motor sizes can be used to produce any amount of torque. For example, in some implementations, the output torque on the rotational joint assembly 310 is greater than or equal to 80 newton meters.

FIG. 3C illustrates a cutaway view of the powered prosthetic joint 300 shown in FIGS. 3A-3B. The motor 102, output sprocket 114 and intermediate shaft 106 can positioned by the actuator housing 200. As shown in FIG. 3C, the output sprocket 114 and motor output shaft 103 can be positioned so that they share the same axis of rotation. As shown in FIG. 3C, the actuator housing 200 can optionally have two pyramidal adapters 302a, 302b attached so that two prosthetics can be attached to the actuator housing 200. The pyramidal adapter 302b can be attached using a rotational joint assembly 310 that is also attached to the output sprocket 114. In the implementation shown in FIG. 3C, the two drive chains 120, 122 are covered by the actuator housing 200. Additionally, a first intermediate sprocket 108 and a second intermediate sprocket 110 can also be covered by the actuator housing 200.

With reference to FIG. 4, implementations of the present disclosure can also be used for prosthetic ankles and feet. According to some implementations of the present disclosure, the same actuator and housing can be used to form different prosthetic joints. For example, the actuator 100 illustrated in FIGS. 1A-2B can be used for both powered prosthetic knees (e.g., the powered prosthetic knee shown in FIGS. 3A-3C) and powered prosthetic ankles and feet (e.g., the prosthetic ankle and foot 400 shown in FIG. 4) or any other prosthetic joint. Implementations of the present disclosure can be connected to different rotational joint assemblies so that they can be connected to different prosthetic body parts. As shown in FIG. 4, the rotational joint assembly 402 is shaped so that it can attach the actuator housing 200 to a flat prosthetic foot plate 404. In the implementation shown in FIG. 4, the actuator housing 200 has a single pyramidal adapter 302a, which can be used to connect the prosthetic ankle and foot 400 to a lower leg prosthetic (not shown).

Implementations of the present disclosure can obtain large ranges of motion using an inexpensive and reliable mechanism. FIG. 5A and 5B illustrate a prosthetic knee 500. In FIG. 5A, the prosthetic knee 500 is in a neutral (not bent) position. In other words, both pyramidal adapters 302a 302b are lined up along the line 502. FIG. 5B illustrates the maximum range of rotation 504 for the example prosthetic knee, which for this non-limiting example implementation is greater than 100 degrees. In the present disclosure, “range of motion” may also be used to refer to the range of rotation 504 of an example prosthetic. It should be understood that embodiments of the present disclosure can rotate further by using different types of rotational joint assembly, and that the amount of rotation, and rotational joint assembly shown in FIG. 5A is intended only as a non-limiting example. For example, some implementations of the present disclosure can include a range of rotation 504 of less than 100 degrees. And, in some implementations the range of rotation 504 can be determined based on the type of prosthesis, and different implementations of the present disclosure can be configured to rotate to different ranges of rotation 504.

### Example Computing Device

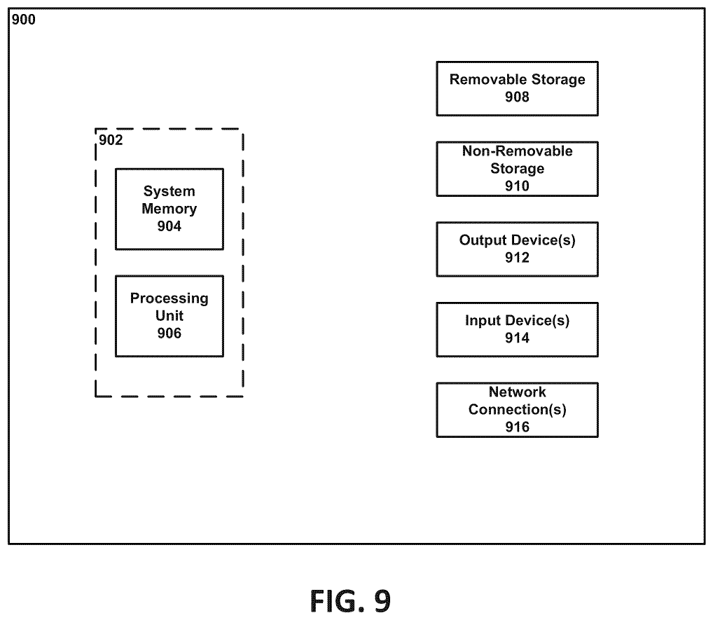

It should be appreciated that the logical operations described herein with respect to the various figures may be implemented (1) as a sequence of computer implemented acts or program modules (i.e., software) running on a computing device (e.g., the computing device described in FIG. 9), (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein.

Referring to FIG. 9, an example computing device 900 upon which the methods described herein may be implemented is illustrated. It should be understood that the example computing device 900 is only one example of a suitable computing environment upon which the methods described herein may be implemented. Optionally, the computing device 900 can be a well-known computing system including, but not limited to, personal computers, servers, handheld or laptop devices, multiprocessor systems, microprocessor-based systems, network personal computers (PCs), minicomputers, mainframe computers, embedded systems, and/or distributed computing environments including a plurality of any of the above systems or devices. Distributed computing environments enable remote computing devices, which are connected to a communication network or other data actuator medium, to perform various tasks. In the distributed computing environment, the program modules, applications, and other data may be stored on local and/or remote computer storage media.

In its most basic configuration, computing device 900 typically includes at least one processing unit 906 and system memory 904. Depending on the exact configuration and type of computing device, system memory 904 may be volatile (such as random access memory (RAM)), non-volatile (such as read-only memory (ROM), flash memory, etc.), or some combination of the two. This most basic configuration is illustrated in FIG. 9 by dashed line 902. The processing unit 906 may be a standard programmable processor that performs arithmetic and logic operations necessary for operation of the computing device 900. The computing device 900 may also include a bus or other communication mechanism for communicating information among various components of the computing device 900.

Computing device 900 may have additional features/functionality. For example, computing device 900 may include additional storage such as removable storage 908 and non-removable storage 910 including, but not limited to, magnetic or optical disks or tapes. Computing device 900 may also contain network connection(s) 916 that allow the device to communicate with other devices. Computing device 900 may also have input device(s) 914 such as a keyboard, mouse, touch screen, etc. Output device(s) 912 such as a display, speakers, printer, etc. may also be included. The additional devices may be connected to the bus in order to facilitate communication of data among the components of the computing device 900. All these devices are well known in the art and need not be discussed at length here.

The processing unit 906 may be configured to execute program code encoded in tangible, computer-readable media. Tangible, computer-readable media refers to any media that is capable of providing data that causes the computing device 900 (i.e., a machine) to operate in a particular fashion. Various computer-readable media may be utilized to provide instructions to the processing unit 906 for execution. Example tangible, computer-readable media may include, but is not limited to, volatile media, non-volatile media, removable media and non-removable media implemented in any method or technology for storage of information such as computer readable instructions, data structures, program modules or other data. System memory 904, removable storage 908, and non-removable storage 910 are all examples of tangible, computer storage media. Example tangible, computer-readable recording media include, but are not limited to, an integrated circuit (e.g., field-programmable gate array or application-specific IC), a hard disk, an optical disk, a magneto-optical disk, a floppy disk, a magnetic tape, a holographic storage medium, a solid-state device, RAM, ROM, electrically erasable program read-only memory (EEPROM), flash memory or other memory technology, CD-ROM, digital versatile disks (DVD) or other optical storage, magnetic cassettes, magnetic tape, magnetic disk storage or other magnetic storage devices.

In an example implementation, the processing unit 906 may execute program code stored in the system memory 904. For example, the bus may carry data to the system memory 904, from which the processing unit 906 receives and executes instructions. The data received by the system memory 904 may optionally be stored on the removable storage 908 or the non-removable storage 910 before or after execution by the processing unit 906.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

### CONCLUSION

It will be understood that each step of a method, block of the flowchart illustrations and/or block diagrams, and combinations of blocks in the flowchart illustrations and/or block diagrams, can be implemented by computer program instructions. These computer program instructions may be provided to a processor of a general-purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

It should be understood that the various techniques described herein may be implemented in connection with hardware or software or, where appropriate, with a combination thereof. Thus, the methods and apparatuses of the presently disclosed subject matter, or certain aspects or portions thereof, may take the form of program code (i.e., instructions) embodied in tangible media, such as floppy diskettes, CD-ROMs, hard drives, or any other machine-readable storage medium wherein, when the program code is loaded into and executed by a machine, such as a computing device, the machine becomes an apparatus for practicing the presently disclosed subject matter. In the case of program code execution on programmable computers, the computing device generally includes a processor, a storage medium readable by the processor (including volatile and non-volatile memory and/or storage elements), at least one input device, and at least one output device. One or more programs may implement or utilize the processes described in connection with the presently disclosed subject matter, e.g., through the use of an application programming interface (API), reusable controls, or the like. Such programs may be implemented in a high level procedural or object-oriented programming language to communicate with a computer system. However, the program(s) can be implemented in assembly or machine language, if desired. In any case, the language may be a compiled or interpreted language and it may be combined with hardware implementations.

While this specification contains many specific implementation details, these should not be construed as limitations on the claims. Certain features that are described in this specification in the context of separate implementations may also be implemented in combination in a single implementation. Conversely, various features that are described in the context of a single implementation may also be implemented in multiple implementations separately or in any suitable subcombination. Moreover, although features may be described above as acting in certain combinations and even initially claimed as such, one or more features from a claimed combination may in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems may generally be integrated together in a single software product or packaged into multiple software products.

It should be appreciated that the logical operations described herein with respect to the various figures may be implemented (1) as a sequence of computer implemented acts or program modules (i.e., software) running on a computing device, (2) as interconnected machine logic circuits or circuit modules (i.e., hardware) within the computing device and/or (3) a combination of software and hardware of the computing device. Thus, the logical operations discussed herein are not limited to any specific combination of hardware and software. The implementation is a matter of choice dependent on the performance and other requirements of the computing device. Accordingly, the logical operations described herein are referred to variously as operations, structural devices, acts, or modules. These operations, structural devices, acts and modules may be implemented in software, in firmware, in special purpose digital logic, and any combination thereof. It should also be appreciated that more or fewer operations may be performed than shown in the figures and described herein. These operations may also be performed in a different order than those described herein. It will be apparent to those skilled in the art that various modifications and variations can be made without departing from the scope or spirit. Other embodiments will be apparent to those skilled in the art from consideration of the specification and practice disclosed herein. It is intended that the specification and examples be considered as exemplary only, with a true scope and spirit being indicated by the following claims.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### Example 1

Development and clinical application of robotic (powered) prostheses is an important advance in the history of lower-limb prosthetics. With actively powered prosthetic joints, a robotic prosthesis may provide significantly improved performance and user experience compared with traditional passive prostheses. A robotic lower-limb prosthesis can be a self-contained portable robotic device with full energy autonomy (i.e., carrying the energy supply onboard). As a human-assistive device, it also can have strict weight and volumetric profile limitations. As such, supplying sufficient joint torque and power under such strict limitations is a major technological challenge in developing such devices. [1, 2]. Despite the high power density of hydraulics, the difficulty of providing a compact hydraulic supply can impair its practicality. With the advances in electronics, computer, and actuation technologies, a variety of robotic lower-limb prostheses have been developed, most of which powered with electric motors. [3-8]. Parallel and serial elastic elements can be used to mimic the ankle biomechanics and improve energy efficiency. [9].

From the actuation technology perspective, some devices use a harmonic drive to obtain large reduction ratio (input speed vs. output speed) within a compact package [11], but this type of sophisticated gearing system is expensive and delicate. The Empower Ankle's ball screw-based actuator, although compact and lightweight, involves two conversions between rotation and linear translation: motor rotation is first converted to ball screw's linear translation, which is then converted to joint rotation through a linkage [6]. Despite the compactness of such actuator design, the back-and-forth conversions between rotation and linear translation result in increased complexity and power loss. Another example powered knee and ankle prostheses uses ball screw-based actuator designs [12, 13]. Yet another example of robotic prostheses includes powered knee and ankle joints where the early versions were pneumatically actuated [14], and later versions use motor power coupled with ball screw or belt/chain-type actuator [16]. The example prostheses can include a 3-stage actuator, with each stage constructed with a timing belt drive [17], where a 3-stage configuration of actuator requires 2 intermediate shafts, which can be difficult to place and increase the heights of these prostheses, limiting the number of amputees that may benefit from the device (i.e., amputees must be tall enough to use them). Implementations of the present disclosure can overcome these challenges.

Implementations of the present disclosure include a dual-stage folded-chain-drive actuator that can reduce the mechanical complexity as well as the weight and/or volumetric profile of a powered prosthetic joint. Compared with 3-stages actuators, the implementation of the present disclosure shown in FIGS. 1A-1B can include a dual-stage design that generates substantial simplification of the mechanical structure: as only one intermediate shaft is required, the actuator can be fully “folded” to obtain an ultra-compact volumetric profile. Each stage of the actuator can be a miniature chain drive, providing inherent flexibility in the power actuator to buffer shock loading and improve comfort in the prosthesis use. Compared with timing belts [17], miniature chains provide higher force capacity and longer life of operation at a much narrower width. As can be seen in FIG. 1A, the width of the entire dual-stage actuator can be even less than the motor itself.

In some implementations, the dual-stage design can allow for the prosthetic joint to incorporate a modern high-performance exterior-rotor brushless DC (ER-BLDC) motor. In an ER-BLDC motor, the magnets in the exterior rotor are distributed at a large diameter, providing a superior torque density (torque output per unit of weight or volume) compared with other DC motors [18-20]. With the high torque output, a small gear ratio can obtain the desired output, significantly reducing the frictional loss and improving the dynamic performance and back-drivability. On the other hand, the unique large-diameter disk-like shape of ER-BLDC motor also poses a challenge for its integration into a prosthetic joint. If placed outside of the rotary joint (e.g., [17]), the excessive height of the device may affect its practical daily use by amputee users. In comparison, the folded-chain-drive configuration of implementations of the present disclosure can incorporate the motor into the rotary joint itself by aligning the output shaft with the motor shaft, producing a highly compact package. Further, using this form factor, the joint can easily reach the desired range of knee flexion (at least 100° for natural sit-to-stand transition [21]) without increasing the device height (FIG. 5B).

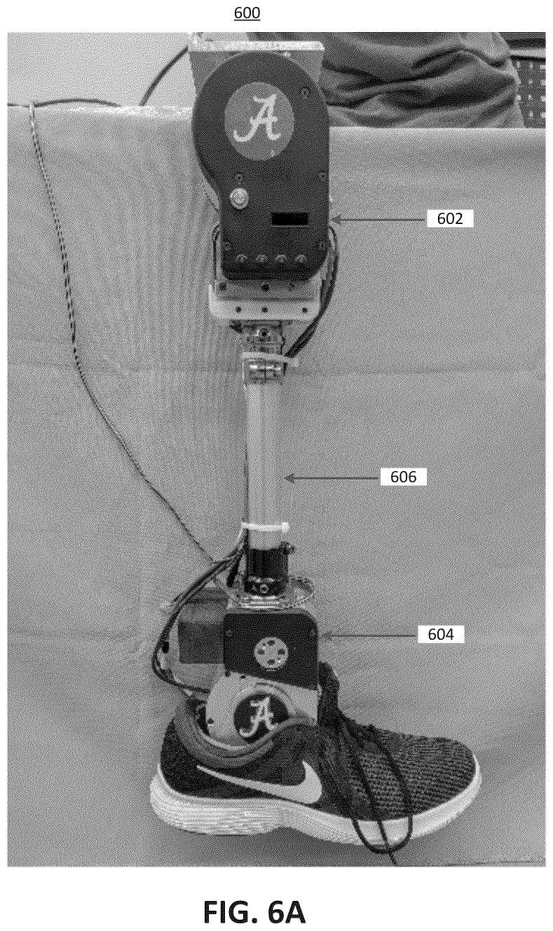

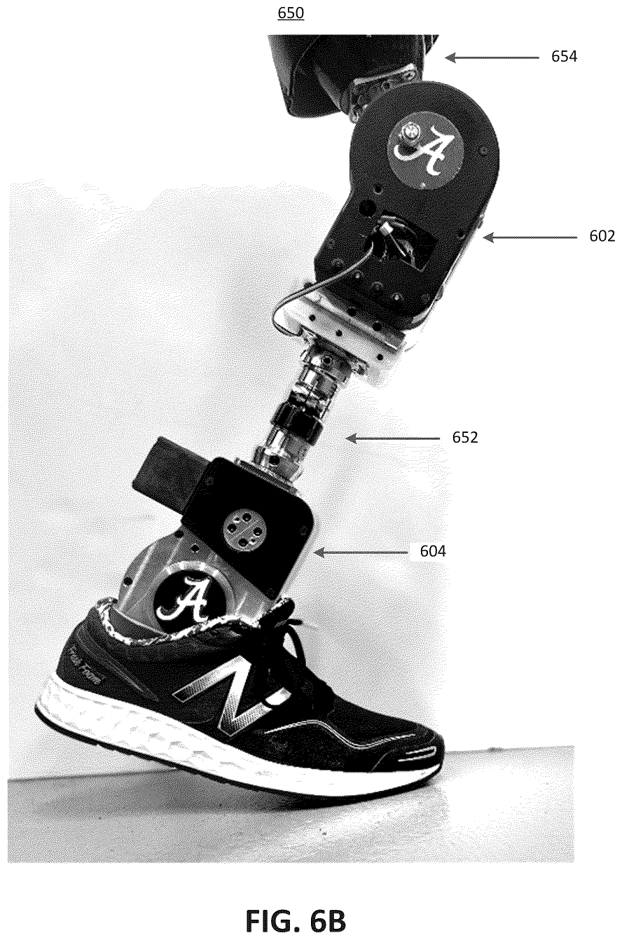

An example implementation of the present disclosure was built and tested, as shown in FIGS. 6A-8. The example implementation is referred to herein as the Common-Core-Components Knee-Ankle Prosthesis (C3KAP). The C3KAP includes a design of the dual-stage folded-chain-drive actuator, combined with an ER-BLDC motor, that can generate a compact, low-height, low-weight, and high-torque prosthetic joint suitable for both knee and ankle joints. FIG. 6A shows the example implementation of a knee prosthesis 602 and ankle prosthesis 604 incorporated into a prosthetic leg 600. The knee prosthesis 602 and ankle prosthesis 604 are by a leg section 606.

FIG. 6B shows another example implementation of the present disclosure that was built and tested. The example implementation 650 includes a knee prosthesis 602 and ankle prosthesis 604 joined together by a leg section 652. The knee prosthesis is also connected to a second leg section 654 that can be worn by a person. Alternatively or additionally, the second leg section 654 can be a prosthetic socket worn over a residual (partial) limb of the person.

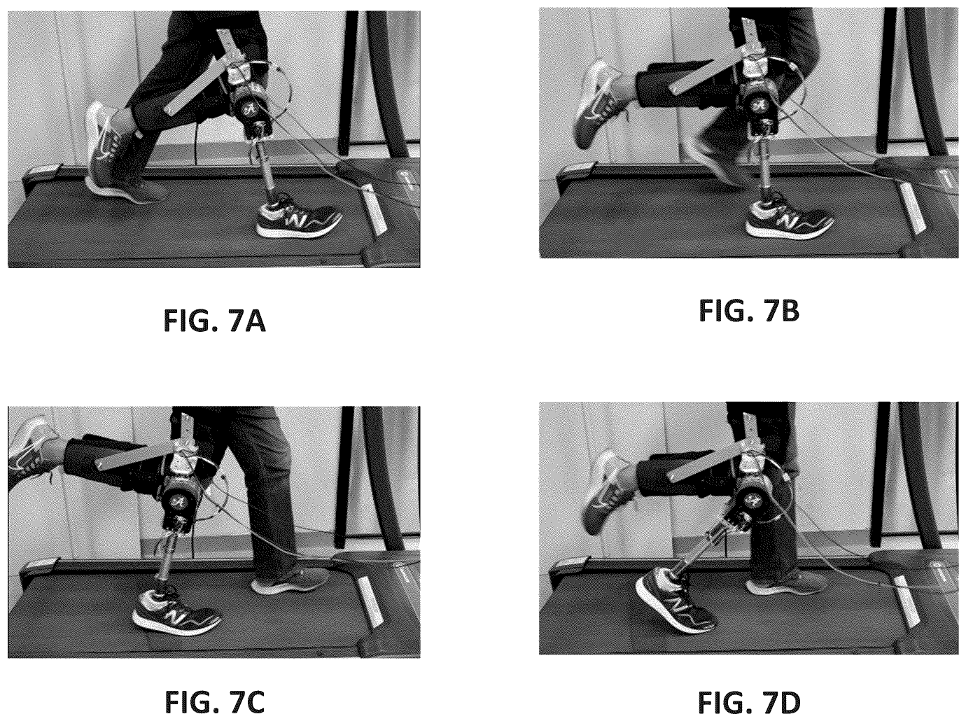

FIGS. 7A-7D show a study performed on the example implementation shown in FIG. 6A. The prosthetic leg was tested by a healthy user. The prosthetic leg was connected to a controller (not shown) that was configured to control the knee and ankle prostheses. Optionally, the controller can include any or all of the components of the computing device 900 shown in FIG. 9.

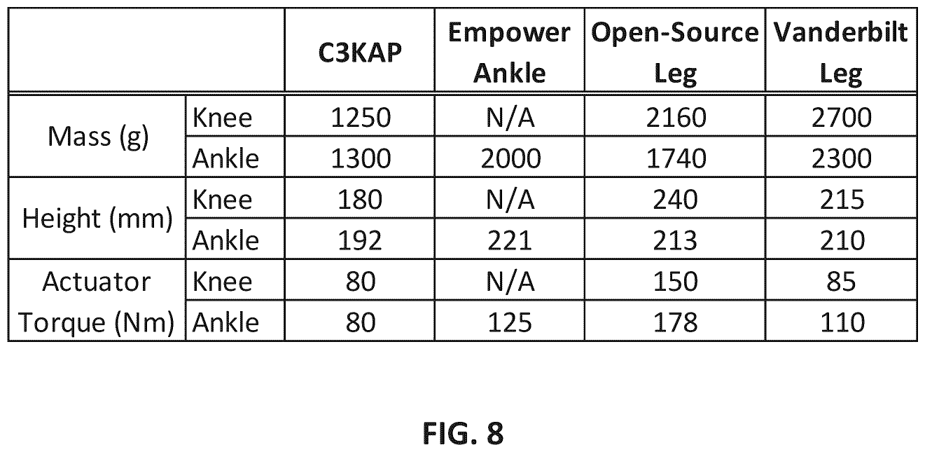

FIG. 8 illustrates experimental results that were collected from the experimental implementations of the prosthetic knee and ankles shown in FIGS. 6A and 6B. The experimental results show that the C3KAP implementations can be lighter weight and shorter than existing prosthetic joint designs. Lightweight prostheses can be less tiring to wear and can require less power to operate. Short prostheses can be desirable for users who have are short and therefore also desire a short prosthesis. The study shows that the combination of features described herein can allow the prosthesis to be effective, despite having a lower actuator torque than some of the alternative designs shown in FIG. 8.

Traditionally, powered knee and powered ankle were treated as completely different prosthetic devices. A prosthetic knee may require a larger range of motion (>100°) than a prosthetic ankle (˜20° [3]), but it can also be relatively taller and wider (similar to the upper part of the human shank). In comparison, a prosthetic ankle must be much narrower (comparable to a biological ankle) and shorter (to fit more amputees). The new design framework, C3KAP can provide a promising solution to fulfill both sets of requirements with a unified basic design, which can be easily configured as a knee or ankle prosthesis with minimal differences. As such, this new design framework may greatly reduce the cost and mechanical complexity of powered lower-limb prostheses. Thus, the example implementation can include powered knee and ankle joints to form a complete lower-limb prosthesis. Additionally, the example implementation can include control circuit boards and batteries attached and covered with 3D-printed plastic covers).

### Example 2

An example robotic transfemoral prosthetic joint was designed and fabricated in a study. The example prosthetic joint can be optionally configured for other joints, including non-limiting examples of prosthetic ankles, elbows, and shoulders. The example prosthetic joint can optionally be used as a system of common components that can be re-used for different prosthetic joints.

The example Knee-Ankle Prosthesis described in the example can provide a compact device that fulfills the shape, size, and weight requirement of an actual healthy knee and ankle. Alternatively or additionally, the example knee-ankle prosthesis, can deliver adequately large torque and power output. In addition to that, providing a low-cost and affordable prosthesis to the amputee is also possible using implementations of the present disclosure.

The study included design of actuators and transmissions. The example knee-ankle prosthesis includes a compact yet powerful actuation mechanism with a high transmission ratio while satisfying the torque-speed requirements. Many powered prosthetic devices utilize electric motors [6]. But to generate acceptable prosthesis behavior [55], an electric motor alone may not be sufficient as electric motors typically generate peak power at high speeds. Although a high gear reductions transmission can address this issue, prosthesis size, weight, efficiency, performance, and electrical systems largely depend on transmission ratios [41]. Therefore, implementations of the present disclosure can create transmissions with lower transmission ratios that can mimic a human lower limb like active prosthesis. Existing electromechanical actuation systems [29-30] [33-35] [39-45] include drawbacks including low output torque, high weight, and/or high complexity.

Series elastic actuators (SEA) can be used for active prostheses. However, the performance of the SEA increases prosthesis weight and reduces control bandwidth [6]. Therefore, the example implementation in the study can be used without SEA. The example implementation of the study can use electromechanical actuators with a low transmission ratio.

In the non-limiting example implementation that was studied, a flat motor (e.g., a multi-pole brushless flat motor) powers the transmission systems. When conducting the detailed design, the power source was chosen first and served as the basis of the transmission design. The example drone motor (U8 Lite-100, TMotor, Jiangxi, China) was selected based on its excellent performance (high torque and power output at a very light weight). While the speed of the motor is relatively low, it is still too high for direct actuation. Subsequently, the transmission of the example implementation was configured to include a two-staged miniature chain drive transmission system considering the chain drive's efficiency and high back drivability. It should be understood that the motor type, torque, and transmission design described herein is intended only as a non-limiting example, and that other gear ratios, sizes, and motor characteristics can be used in implementations of the present disclosure.

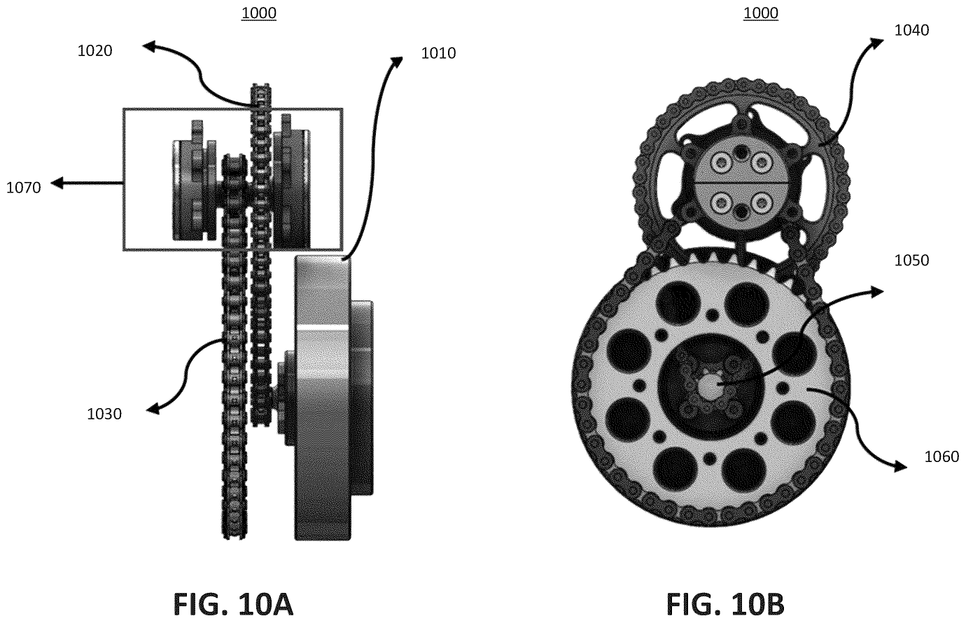

With reference to FIG. 10A and FIG. 10B, an example transmission system 1000 is shown that was studied. The transmission system includes a brushless DC motor 1010, an RS 15 size chain 1020, a RS 25 size chain 1030, a 42-teethRS 15 size sprocket and 8 teeth-RS 25 size sprocket 1040, a 7-teeth-RS 15 size sprocket 1050, a 39 teeth-RS 25 size sprocket 1060. The transmission can further include a complete eccentric mount 1070.

The design parameters of the example actuation system were determined as follows: the motor shaft is directly coupled to a 7-teeth small sprocket (e.g., 7-teeth-RS 15 size sprocket 1050 shown in FIG. 10A), which drives a 42-teeth sprocket supported by the aforementioned intermediate shaft through an RS15 roller chain: on the same shaft, the motion is transmitted to an 8-teeth sprocket (e.g., 42-teethRS 15 size sprocket and 8 teeth-RS 25 size sprocket 1040), which drives a 39-teeth sprocket (e.g., a 39 teeth-RS 25 size sprocket 1060) through RS25 roller chains. Such a two-stage design provides a total transmission ratio of almost 29.25:1, and thus the output joint torque reaches as high as 80 N-m. Such high torque capacity forms a basis for implementing a stable walking control algorithm, and the performance of the prostheses. This available torque value is more than the required torque for knee joints of 100 kg human but may be lower than required torque for ankle joints of 100 kg amputee. Although for an amputee weighing less than 50 kg, this torque capacity can be enough. Again, the transmission ratios, sprocket sizes, and forces described herein are intended only as non-limiting examples.

Implementations of the present disclosure can address the torque issues with 100 kg amputee. Implementations of the present disclosure can include a carbon fiber-reinforced polymer (CFRP) glass fiber spring foot (Energy Storing and Return Foot) instead of a solid ankle cushion heel (SACH) foot. This type of energy storing and return foot can reduce the actuation torque requirements of the device by adding approximately 90 Nm torque (based on this specific design criteria) during push-off. This can solve the low ankle joint torque issues.

The study further included chain drive design and analysis for the example implementation of the present disclosure. As described herein, keeping the transmission ratio low can have several benefits like low prosthetic joint weight, compact size and shape, and less electrical demands. As the chain drive provides the flexibility feature of a belt drive and positive engagement benefits of gear, it can be a very efficient choice for low-speed and high torque applications [59]. Moreover, unlike the belt, it can have a minimum chance of slippage and requires only a slight adjustment. In addition to that, there is flexibility in shaft center to center distances, unlike gear drives. These characteristics can make chain drive a better choice over other types of transmission systems. Therefore, a chain drive mechanism with only a 29.25:1 transmission ratio is selected, which is sufficient to fulfill the requirements of prosthetic joint design. A standard two-stage configuration can be enough, but this increases the prosthetic device's length. In some cases, long prosthetics may only be usably by individuals with longer limbs (e.g., taller patients can use longer prosthetic legs). Thus, it is beneficial to keep the length of the prosthetic short to allow a prosthetic device to be used by amputees with shorter limbs (e.g., shorter patients can desire shorter prosthetic legs). Thus, implementations can include drive systems that optimize the use of length and width so that the prosthetic is suitable for a wider range of amputees. The example implementation includes a folded two-stage chain drive configuration that can result in a shorter prosthetic that can be used by a wide range of amputees for different joints. The example implementation is shown and described herein with reference to FIG. 1B.

The example implementation further included structural housing design and analysis. For the example implementation, a clamshell type design was selected as housing for prosthesis components. The shape of the clamshell configuration was designed to consider the the high range of motion of the knee (e.g., 120 degrees) because if the shape of the clamshells is enough to incorporate knee range of motion, it can also be enough for ankle joints due to its low range of motion (45 degrees) requirements compared to knee joints. Therefore, the goal was to choose a shape such that it is possible to achieve knee range of motion without any interferences, can house all transmission-actuation components, and make a similar shape of the prosthesis joints as biological lower limb joints. FIGS. 5A and 5B illustrate an example range of motion of the housing.

In addition to the shape of the clamshell, a three-part clamshell was selected, considering that the chain sprocket should be pre-assembled before placing it into the clamshell parts. Optionally, a two-part clamshell configuration can lower the number of structural elements but may increase the difficulty of assembling the chain drive assembly, depending on the flexibility of

[symbol]

The clamshell parts are central part, idle support, and output shell, which are also referred to herein as the central shell 204, motor side shell 206, and output side shell 202. Optionally, the clamshell parts can be machined from 7075-T6 Aluminum, an aerospace-grade material. This example material was chosen due to its capacity to withstand significant loading conditions, and it is much lighter than steel and other structural materials. As keeping the prosthetic joint weight as minimum as possible was a design goal of the study, this 7075-T6 Aluminum is suitable for the example prosthetic joints. Finite element analysis was performed on each shell piece to check its structural durability under the load of 100 kg human. Another major structural part which sits on the positioning feature created on top of the three shell pieces. Optionally, this can be formed as a separate piece so that the load cell can be mounted on top of that piece without removing whole shells, and this part makes it easier to inspect the chain for proper tensioning, assembling the last stage of the chain drive, and which can be also used to lubricate the chain if required.

For user safety, the housing can optionally incorporate four bumpers/hard stops and two bumpers/hard stops in the knee joint and ankle joint, respectively. The bumpers can optionally stop the motion of the joints beyond the dedicated range of motion (ROA) of knee and ankle joints. Although ROA can optionally be specified in the control algorithm, the bumpers can optionally be used as an extra feature to further improve the amputees' safety and well-being.

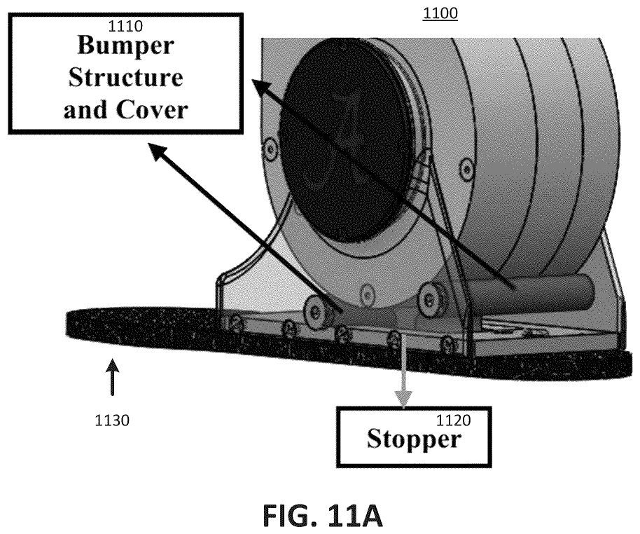

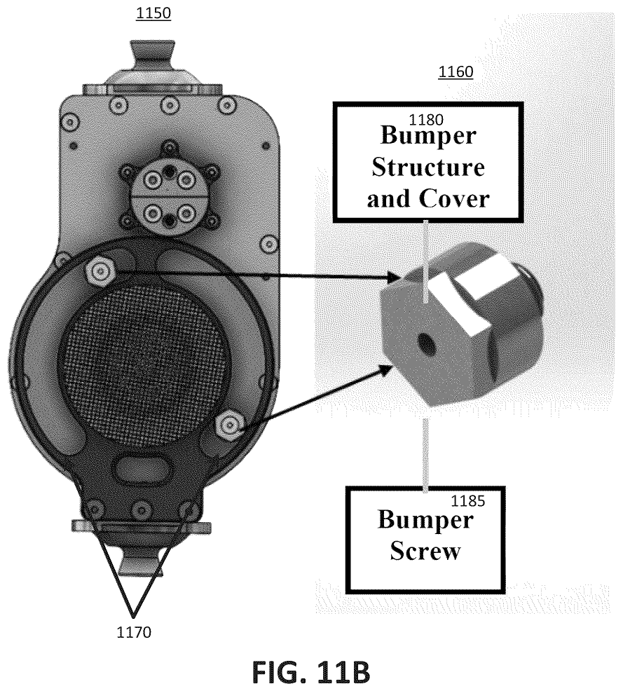

FIG. 11A illustrates an example housing 1100 including two bumpers 1110. The bumpers 1110 can optionally be covered in a bumper cover, as shown in FIG. 11A. A stopper 1120 can be positioned between the two bumpers 1110 to limit the range of motion of the prosthetic 1130. The prosthetic 1130 in FIG. 11A is a prosthetic foot, but it should be understood that implementations of the present disclosure can include any arrangement of any number of bumpers 1110 and stoppers 1120 to limit the range of motion of any prosthetic. For example, according to implementations of the present disclosure a prosthetic knee or arm can optionally use bumpers that are spread further apart to increase the range of motion. Alternatively, other prosthetics can include bumpers that are positioned closer together to limit the range of motion to less than that shown in FIG. 11A.

FIG. 11B illustrates an example prosthetic knee 1150. The example prosthetic knee 1150 includes two bumpers 1160 configured to fit in two tracks 1170. The bumpers 1160 limit the movement of the prosthetic knee 1150 to the range of motion of the two tracks 1170. The bumpers 1160 can optionally include bumper structures 1180 that can optionally be covered (not shown) and a bumper screw 1185 that can be used to attach the bumper 1160 to the prosthetic knee 1150. It should be understood that the dimensions, number, and arrangement of bumpers 1160 and tracks 1170 can be modified to attain different ranges of motion for different prosthetics, including prosthetics other than prosthetic knees. It should also be understood that the bumpers 1160 can be attached using attachments other than screws.

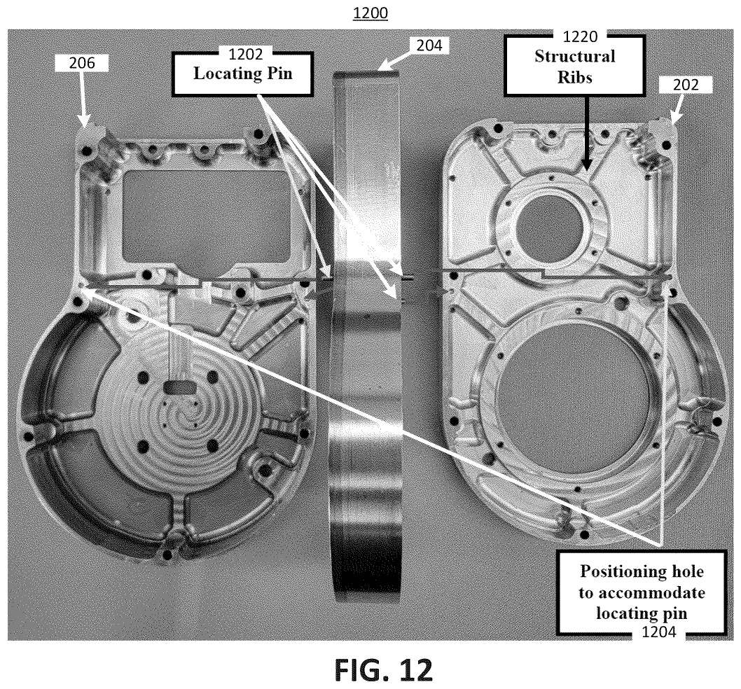

FIG. 12 illustrates an example clamshell housing 1200 for an implementation of the present disclosure that was studied. The example clamshell housing includes locating pins 1202 and positioning holes 1204 sized to accommodate the locating pins 1202. Locating pins 1202 can be used to avoid improper alignment of the shell pieces during assembly. While FIG. 12 shows three locating pins 1202 and positioning holes 1204, it should be understood that any number of locating pins 1202 and/or positioning holes 1204 can be used in different implementations of the present disclosure. Additionally, in some implementations, a cross-roller bearing can optionally be used to support the axial/radial loads and the bending moments in the output side. The clamshell housing 1200 can optionally include structural ribs 1220 formed in any or all of the output side shell 202, central shell 204, and motor side shell 206. The output side shell 202, central shell 204, and motor side shell 206 are described in greater detail herein with reference to FIG. 2A.

Optionally, all moving transmission parts and motor can be entirely enclosed in the clamshell housing 1200. Enclosing the transmission parts and motor completely (or partially) can improve the user's safety and/or protect the transmission components and motor from dirt and debris.

### References

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

### REFERENCES

1. Flowers, W.C. and R. W. Mann, An electrohydraulic knee-torque controller for a prosthesis simulator. Journal of biomechanical engineering, 1977. 99(1): p. 3-8.
2. Flowers, W.C., A man-interactive simulator system for above-knee prosthetics studies. 1973, Massachusetts Institute of Technology.
3. Cutti, A., et al., The effects of the ‘Power Knee’ prosthesis on amputees metabolic cost of walking and symmetry of gait—Preliminary results. Gait & Posture, 2008(28): p. S38.
4. Herr, H.M. and A.M. Grabowski, Bionic ankle-foot prosthesis normalizes walking gait for persons with leg amputation. Proceedings of the Royal Society B-Biological Sciences, 2012. 279(1728): p. 457-464.
5. Au, S.K., J. Weber, and H. Herr, Powered ankle-foot prosthesis improves walking metabolic economy. IEEE Transactions on Robotics, 2009. 25(1): p. 51-66.
6. Au, S.K. and H.M. Herr, Powered ankle-foot prosthesis. IEEE Robotics & Automation Magazine, 2008. 15(3): p. 52-59.
7. Au, S., M. Berniker, and H. Herr, Powered ankle-foot prosthesis to assist level-ground and stair-descent gaits. Neural Networks, 2008. 21(4): p. 654-666.
8. Au, S.K., et al. Powered ankle-foot prosthesis for the improvement of amputee ambulation. in 2007 29th annual international conference of the IEEE engineering in medicine and biology society. 2007. IEEE.
9. Ottobock. Empower|Ottobock US. [cited 2022 July 1]: Available from: https://www.ottobockus.com/products/empower-ankle/.
10. Ossur PowerKnee: Instruction for Use. 2018.
11. Tuttle, T.D. and W.P. Seering, A nonlinear model of a harmonic drive gear transmission. IEEE Transactions on Robotics and Automation, 1996. 12(3): p. 368-374.
12. Gabert, L., et al., A compact, lightweight robotic ankle-foot prosthesis: Featuring a powered polycentric design. IEEE robotics & automation magazine, 2020. 27(1): p. 87-102.
13. Lenzi, T., et al., Design, development, and testing of a lightweight hybrid robotic knee prosthesis. The International Journal of Robotics Research, 2018. 37(8): p. 953-976.
14. Sup, F., A. Bohara, and M. Goldfarb, Design and Control of a Powered Transfemoral Prosthesis. The International Journal of Robotics Research, 2008. 27(2): p. 263-273.
15. Sup, F., et al., Preliminary evaluations of a self-contained anthropomorphic transfemoral prosthesis. IEEE/ASME Transactions on mechatronics, 2009. 14(6): p. 667-676.
16. Lawson, B.E., et al., A Robotic Leg Prosthesis Design, Control, and Implementation. Ieee Robotics & Automation Magazine, 2014. 21(4): p. 70-81.
17. Azocar, A.F., et al., Design and clinical implementation of an open-source bionic leg. Nature biomedical engineering, 2020. 4(10): p. 941-953.
18. Seok, S., et al. Actuator design for high force proprioceptive control in fast legged locomotion. in 2012 IEEE/RSJ International Conference on Intelligent Robots and Systems. 2012. IEEE.
19. Seok, S., et al., Design principles for energy-efficient legged locomotion and implementation on the MIT cheetah robot. Ieee/asme transactions on mechatronics, 2014. 20(3): p. 1117-1129.
20. Lee, U.H., C.- W. Pan, and E.J. Rouse. Empirical characterization of a high-performance exterior-rotor type brushless DC motor and drive. in 2019 IEEE/RSJ International Conference on Intelligent Robots and Systems (IROS). 2019. IEEE.
21. Mak, M.K., et al., Joint torques during sit-to-stand in healthy subjects and people with Parkinson's disease. Clinical Biomechanics, 2003. 18(3): p. 197-206.

1. An actuator for a powered prostheses comprising:
a motor comprising a motor output shaft, connected to a motor output sprocket:
an intermediate shaft comprising a first intermediate sprocket and a second intermediate sprocket:
an output sprocket:
a first drive chain configured to engage the first intermediate sprocket and the motor output sprocket; and
a second drive chain configured to engage the second intermediate sprocket and the output sprocket.

2. The actuator of claim 1, further comprising an actuator housing, wherein the motor is fixedly attached to the actuator housing.

3. The actuator of any claim 2, wherein the actuator housing comprises a proximal end and a distal end, the proximal end being proximal to the output sprocket, wherein the distal end comprises an attachment surface.

4. The actuator of claim 3, wherein the proximal end comprises a rotational joint assembly.

5. The actuator of claim 1, wherein the output sprocket defines a first axis of rotation and the motor output sprocket defines a second axis of rotation, and wherein the first axis of rotation is coaxial with the second axis of rotation.

6. The actuator of claim 1, wherein the motor is an exterior-rotor brushless DC (ER-BLDC) motor.

7. The actuator of claim 1, wherein output sprocket produces less than or equal to 80 newton meters of torque.

8. The actuator of claim 1, wherein the actuator comprises a dual-stage actuator.

9. The actuator of claim 1, wherein the actuator further comprises a prosthesis.

10. The actuator of claim 9, wherein the prostheses is a prosthetic ankle.

11. The actuator of claim 9, wherein the powered prostheses is a prosthetic knee.

12. The actuator of claim 9, wherein the actuator is less than 200 mm long.

13. The actuator of claim 1, wherein the actuator has a range of motion greater than 100 degrees.

14. A powered prosthesis comprising:
a first limb section and a second limb section:
an actuator coupled to the first limb section and the second limb section, the actuator comprising:
a motor comprising a motor output shaft and a motor output sprocket:
an intermediate shaft comprising a first intermediate sprocket and a second intermediate sprocket:
an output sprocket:
a first drive chain configured to engage the first intermediate sprocket and the motor output sprocket: and
a second drive chain configured to engage the second intermediate sprocket and the output sprocket.

15. The powered prosthesis of claim 14, wherein the output sprocket defines a first axis of rotation and the motor output sprocket defines a second axis of rotation, and wherein the first axis of rotation is coaxial with the second axis of rotation.

16. The powered prosthesis of claim 14, wherein output sprocket produces less than or equal to 80 newton meters of torque.

17. The powered prosthesis of claim 14, wherein the actuator is a dual-stage actuator.

18. The powered prosthesis of claim 14, wherein the first limb section comprises a lower prosthetic leg, and the second limb section comprises a prosthetic foot.

19. The powered prosthesis of claim 14, wherein the first limb section and the second limb section have a range of motion greater than 100 degrees.

20. The powered prosthesis of claim 14, wherein the actuator is less than 200 mm long.
